(19) United States
(12) Patent Application Publication
(10) Pub. No.: US 2021/0304460 A1
(43) Pub. Date: Sep. 30, 2021

(54) METHOD AND APPARARUS FOR GENERATING SYNTHETIC 2D IMAGE

(71) Applicant: VIEWORKS CO., LTD., Anyang-si (KR)

(72) Inventor: Young Joo HAN, Uiwang-si (KR)

(73) Assignee: VIEWORKS CO., LTD., Anyang-si (KR)

(21) Appl. No.: 17/215,797

(22) Filed: Mar. 29, 2021

(30) Foreign Application Priority Data
Mar. 30, 2020 (KR) 10-2020-0037922

(51) Int. Cl. G06T 11/00; G06T 7/00; G06T 7/11; A61B 6/02; A61B 6/00

(52) U.S. Cl. CPC G06T 11/006; G06T 7/0012; G06T 2207/30068; A61B 6/025; A61B 6/502; G06T 7/11

(57) ABSTRACT

Provided is an apparatus for generating a synthetic 2D image, which includes: an image input unit receiving a 2D image at a plurality of angles or locations for a target object from a detector of a radiographic image acquisition apparatus; a tomosynthesis unit generating a 3D image reconstructed by using the 2D image input into the image input unit; a segmentation map generation unit generating a 3D segmentation map including segmentation data indicating characteristics or types of voxels constituting the 3D image; and a synthetic 2D image synthesis unit generating a synthetic 2D image by using the reconstructed 3D image and the 3D segmentation map.

### CROSS-REFERENCE TO RELATED APPLICATIONS

This application claims priority to and the benefit of Korean Patent Application No. 10-2020-0037922 filed in the Korean Intellectual Property Office on Mar. 30, 2020, the entire contents of which are incorporated herein by reference.

### TECHNICAL FIELD

The present invention relates to a method and an apparatus for generating a synthetic 2D image. More particularly, the present invention relates to a method and an apparatus for generating a synthetic 2D image, which generate and provide a synthetic 2D image by using a semantic segmentation technique.

### BACKGROUND ART

Tomosynthesis is a technique for generating a 3D image based on 2D image data captured at a limited angle. The generated 3D image is advantageous in that it is possible to know the structure of a subject that is lost as data is superimposed in the conventional 2D image. In addition, compared to computed tomography (CT), the tomosynthesis has an advantage of taking less time to capture and effectively obtaining an image of a desired cross section with only a relatively small number of images.

However, there is an inconvenience that in order to perform diagnosis by extracting an image of a desired cross section from the generated 3D image, a user should perform the diagnosis while directly adjusting a depth. For this, a synthetic 2D technology that generates and provides a new 2D image to the user by using only valid information in 3D data or technologies that display a valid slice among 3D image data are developed. However, the existing method is difficult to be flexibly applied to other application technologies, and has limitations, such as being limited to the purpose of simply displaying an image.

### SUMMARY OF THE INVENTION

The present invention has been made in an effort to provide a method and an apparatus for generating a synthetic 2D image, which generate a synthetic 2D image by using a semantic segmentation technique in order to facilitate diagnosis and analysis.

An exemplary embodiment of the present invention provides an apparatus for generating a synthetic 2D image, which includes: an image input unit receiving a 2D image at a plurality of angles or locations for a target object from a detector of a radiographic image acquisition apparatus; a tomosynthesis unit generating a reconstructed 3D image by using the 2D image input into the image input unit; a segmentation map generation unit generating a 3D segmentation map including segmentation data indicating characteristics or types of voxels constituting the 3D image; and a synthetic 2D image synthesis unit generating a synthetic 2D image by using the reconstructed 3D image and the 3D segmentation map.

In an exemplary embodiment, the synthetic 2D image synthesis unit may generate a synthetic 2D image in which the intensity of at least one material of the target object is adjusted by using the segmentation data.

In an exemplary embodiment, the segmentation map generation unit may assign a class label as the segmentation data to the voxel.

The segmentation map generation unit may receive the 2D image from the image input unit and acquire a plurality of 2D segmentation data by performing semantic segmentation in the 2D image, and generate the 3D segmentation map by using the plurality of 2D segmentation data.

The segmentation map generation unit may generate the 3D segmentation map by back-projecting the plurality of 2D segmentation data.

The segmentation map generation unit may perform the semantic segmentation for each class label for the 2D image to acquire 2D segmentation data for each class label.

In an exemplary embodiment, the segmentation map generation unit may generate the 3D segmentation map by performing the semantic segmentation for each voxel of the reconstructed 3D image.

In an exemplary embodiment, the apparatus may further include a semantic filter unit generating a semantic filter as a weight to be applied to the segmentation data of the voxel included in the 3D segmentation map.

The weight may be set separately for each segmentation data or for each voxel.

The weight may be generated through a normalization process.

The synthetic 2D image synthesis unit may generate the synthetic 2D image by multiplying the intensity of the voxel by the semantic filter.

Another exemplary embodiment of the present invention provides a method for generating a synthetic 2D image, which includes: (a) receiving a 2D image at a plurality of angles or locations for a target object from a detector of a radiographic image acquisition apparatus; (b) generating, by a tomosynthesis unit, a reconstructed 3D image by using the 2D images; (c) generating, by a segmentation map generation unit, a 3D segmentation map including segmentation data indicating characteristics or types of voxels of the reconstructed 3D image; and (d) generating, by a synthetic 2D image synthesis unit, a synthetic 2D image by using the reconstructed 3D image and the 3D segmentation map.

In an exemplary embodiment, in step (d), a synthetic 2D image may be generated in which the intensity of at least one material of the target object is adjusted by using the segmentation data.

In step (c), the segmentation map generation unit may receive the 2D image from the image input unit and acquire a plurality of 2D segmentation data by performing semantic segmentation in the 2D image, and generate the 3D segmentation map by using the plurality of 2D segmentation data.

In step (c), the 3D segmentation map may be generated by performing the semantic segmentation for each voxel of the reconstructed 3D image. Further, in step (d), a semantic filter as a weight to be applied to segmentation data of each voxel of the 3D segmentation map may be generated and the synthetic 2D image may be generated by using the semantic filter.

The weight may be set separately for each segmentation data or for each voxel.

The weight may be generated through a normalization process.

In an exemplary embodiment, in step (d), the synthetic 2D image may be generated by multiplying the intensity of the voxel by the semantic filter.

According to an exemplary embodiment of the present invention, when a synthetic 2D image is generated from a 3D image generated by tomosynthesis, intensity of a specific material or a part having a characteristic in an image is adjusted according to a request or setting of a user to provide an adaptive synthetic 2D image.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram illustrating an example of a radiographic image acquisition apparatus.

FIG. 2 is a block diagram illustrating a configuration of an apparatus for generating a synthetic 2D image according to an exemplary embodiment of the present invention.

FIG. 3 is a diagram for describing a process of generating a segmentation map by a segmentation map generation unit in an apparatus for generating a synthetic 2D image according to an exemplary embodiment of the present invention.

FIG. 4 is a diagram exemplarily illustrating a process of generating a segmentation map by a segmentation map generation unit in an apparatus for generating a synthetic 2D image according to an exemplary embodiment of the present invention.

FIG. 5 is a diagram illustrating that a synthetic 2D image is generated by applying a semantic filter in an apparatus for generating a synthetic 2D image according to an exemplary embodiment of the present invention.

FIG. 6 is a diagram for describing that a synthetic 2D image is generated by applying a semantic filter in an apparatus for generating a synthetic 2D image according to an exemplary embodiment of the present invention.

FIG. 7 is a diagram exemplarily illustrating that a synthetic 2D image is generated by an apparatus for generating a synthetic 2D image according to an exemplary embodiment of the present invention.

FIG. 8 is a flowchart illustrating a method for generating a synthetic 2D image according to an exemplary embodiment of the present invention.

FIG. 9 is a flowchart illustrating a method for generating a synthetic 2D image according to another exemplary embodiment of the present invention.

FIG. 10 is a diagram schematically illustrating a method for generating a synthetic 2D image according to an exemplary embodiment of the present invention.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter, a preferred embodiment of the present invention will be described in detail with reference to the accompanying drawings. First, when reference numerals refer to components of each drawing, it is to be noted that although the same components are illustrated in different drawings, the same components are denoted by the same reference numerals as possible. Further, in describing the present invention, a detailed description of known related configurations and functions may be omitted to avoid unnecessarily obscuring the subject matter of the present invention. Further, hereinafter, the preferred embodiment of the present invention will be described, but the technical spirit of the present invention is not limited thereto or restricted thereby and the embodiments can be modified and variously executed by those skilled in the art.

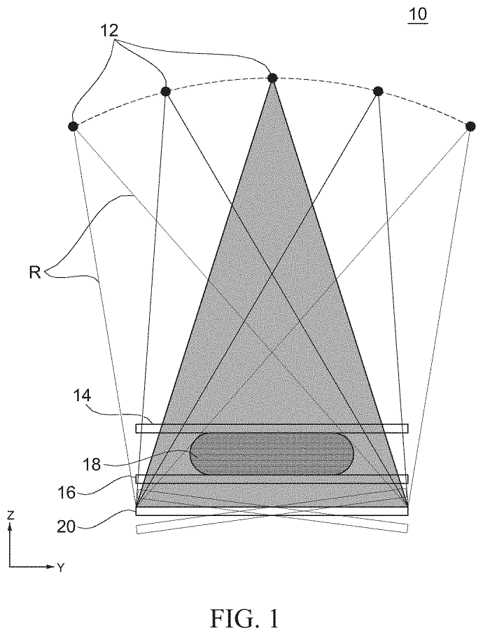

FIG. 1 is a diagram illustrating an example of a radiographic image acquisition apparatus.

The radiographic image acquisition apparatus 10 includes a radiation source 12 irradiating radiation R to a target object 18, and a detector 20 detecting the radiation R that is emitted from the radiation source 12 and transmitted through the target object 18. In an exemplary embodiment, the radiation R may be an X-ray, but the radiation R is not limited to the X-ray and may include other radiation such as a gamma ray that may be applied to a medical image. Further, in an exemplary embodiment, the target object 18 may be a breast, and the radiographic image acquisition apparatus 10 may further include a support plate 16 supporting the breast and a compression paddle 14 compressing the breast from the top (for reference, in FIG. 1, a vertical direction is indicated by a Z axis, a horizontal direction is indicated by a Y axis, and a direction perpendicular to the drawing becomes an X axis).

At least one of the radiation source 12 or the detector 20 of the radiographic image acquisition apparatus 10 may be rotated or repositioned at a predetermined angle with respect to the target object 18 and the detector 20 may acquire a 2D image for the target object 18 at a plurality of angles or locations. In an exemplary embodiment, the detector 20 is implemented in a digital scheme to acquire 2D projection data for the target object 18. In an exemplary embodiment, image data acquired by the radiographic image acquisition apparatus 10 may be used for Digital Breast Tomosynthesis (DBT).

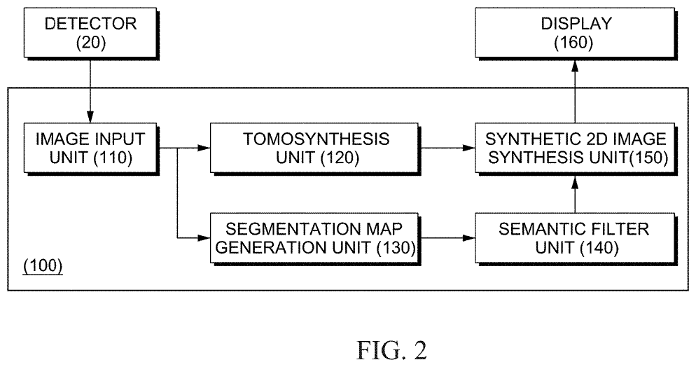

FIG. 2 is a block diagram illustrating a configuration of an apparatus for generating a synthetic 2D image according to an exemplary embodiment of the present invention.

The apparatus 100 for generating a synthetic 2D image according to an exemplary embodiment of the present invention may include an image input unit 110 receiving a plurality of 2D images, which are 2D projection data, from the detector 20 of the radiographic image acquisition apparatus 10, a tomosynthesis unit 120 reconstructing a 3D image using the image input through the image input unit 110, a segmentation map generation unit 130 generating a 3D segmentation map by using the image input into the image input unit 110, and a synthetic 2D image synthesis unit 150 generating a synthetic 2D image by using the 3D image reconstructed by the tomosynthesis unit 120 and the 3D segmentation map generated by the segmentation map generation unit 130. Further, the apparatus 100 for generating a synthetic 2D image may further include a semantic filter unit 140 generating a semantic filter for generating the synthetic 2D image according to the setting or request of the user. The semantic filter may be used for adaptively applying the 3D segmentation map generated by the segmentation map generation unit 130.

Meanwhile, the synthetic 2D image generated by the synthetic 2D image synthesis unit 150 may be presented to a user through a display 160. Further, the user may input setting or request for generating the synthetic 2D image through a user interface (not illustrated) such as a mouse, a keyboard, or a touch pad.

The image input unit 110 receives the plurality of 2D images which are the 2D projection data from the detector 20 of the radiographic image acquisition apparatus 10. The image input unit 110 may be configured to perform preprocessing such as noise removal or signal amplification for the 2D image transferred from the detector 20.

The tomosynthesis unit 120 applies a tomosynthesis technique to the plurality of 2D images transferred from the image input unit 110 and reconstructs the 3D image including the target object 18.

The segmentation map generation unit 130 generates the 3D segmentation map for a region including the target object 18. The 3D segmentation map may be appreciated as a set of voxel information on 3D classified according to characteristics or physical properties of the target object 18. Specifically, a class label for classifying the characteristic or type of each voxel of the reconstructed 3D image may be granted for each voxel and a set of class labels of the respective voxels may be the 3D segmentation map.

In an exemplary embodiment, the segmentation map generation unit 130 performs semantic segmentation for each of the plurality of 2D images transferred from the image input unit 110 to acquire 2D segmentation data and generates the 3D segmentation map from the 2D segmentation data.

Of course, it may be possible for the segmentation map generation unit 130 to directly generate the 3D segmentation data by using 3D segmentation, but it may be difficult to implementation the synthesis due to various problems. In the 3D data reconstructed by using the tomosynthesis, there is a ghost image phenomenon that a voxel in which data is deficient is influenced by surrounding voxels as a 2D image captured at a limited angle is used. A ghost image refers to a phenomenon that the corresponding voxel has incorrect information due to insufficient data for reconstruction and due to effect of the surrounding voxels. Since the 3D data shows a different pattern from general 3D data due to the ghost image, a segmentation result may not be good. Further, since the size of the 3D data may be several tens of times or hundreds of times larger than that of the 2D data, there may be a physical limitation in performing the semantic segmentation requiring a large amount of computation.

In an exemplary embodiment of the present invention, there is no limitation on the method for generating the 3D segmentation map by the segmentation map generation unit 130. However, when the limitation due to such a reason is expected, as described above, the segmentation map generation unit 130 performs the semantic segmentation for the plurality of 2D images to acquire 2D segmentation data and reconstructs the 2D segmentation data in 3D to generate the 3D segmentation map.

In order to facilitate the description of the present invention, the symbols of data or images in each step are defined as shown in Table 1.

TABLE 1

| Symbols | Description |
|---|---|
| Tₚ | 2D image (2D projection data) for the target object 18 |
| | acquired by the radiographic image acquisition apparatus |
| | 10 for tomosynthesis |
| Tᵣ | 3D image (data) generated and reconstructed by using a |
| | plurality of 2D images Tₚ |
| S_{p,} | 2D segmentation data generated by segmenting the 2D image |
| S_{p(label)} | Tₚ. A label is added and displayed for a specific class |
| | label. |
| S_{r,} | 3D segmentation data reconstructed for each label generated |
| S_{r(label)} | by using a plurality of 2D segmentation data Sₚ. The label |
| | is added and displayed for a specific class label. |
| Sᵣₑₚ | 3D segmentation map. As an example, a plurality of S_{r(label)} is |
| | generated |
| F_{syn} | Synthetic 2D image generated by using the reconstructed 3D |
| | image Tᵣ and the 3D segmentation map Sᵣₑₚ |

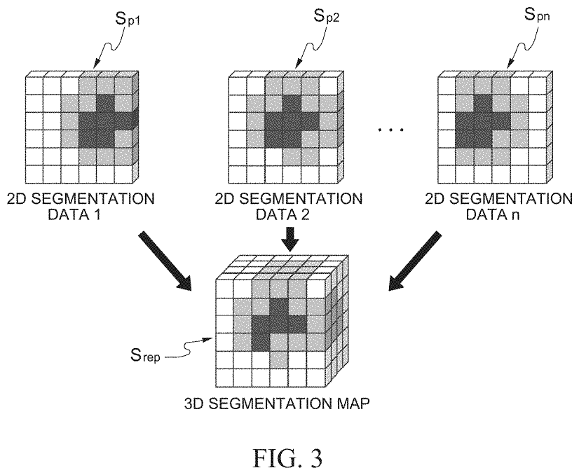

FIG. 3 is a diagram for describing a process of generating a segmentation map by a segmentation map generation unit in an apparatus for generating a synthetic 2D image according to an exemplary embodiment of the present invention.

The segmentation map generation unit 130 performs semantic segmentation for each of the plurality of 2D images Tₚ transferred from the image input unit 110 to acquire 2D segmentation data 1 to n (Sₚ₁, Sₚ₂, . . . , Sₚₙ). The semantic segmentation means that the class label is granted to each pixel in the 2D image Tₚ. The class label in the 2D image Tₚ including the target object 18 may be a bone, tissue, and air. Further, when the target object 18 is the breast, the class label may include a mammary gland. In an exemplary embodiment, the semantic segmentation for the 2D image Tₚ may be performed by using an artificial neural network such as a deep learning based algorithm. Further, it may also be possible to perform the semantic segmentation by using a technique such as K-means clustering, otsu thresholding, or fuzzy clustering.

Meanwhile, in the case of the 2D image acquired by the detector 20 of the radiographic image acquisition apparatus 10, various class labels may be granted to one pixel unlike a general image acquired by a camera, etc., due to characteristics of the radiation that passes through an object. That is, at least two class labels among class labels such as the bone, the tissue, the air, and the mammary gland may be granted to one pixel. As a result, multi-label pixelwise classification may be performed for each of the plurality of 2D images and the plurality of 2D segmentation data Sₚ₁, Sₚ₂, . . . , Sₚₙ may be acquired.

The segmentation map generation unit 130 generates the 3D segmentation map Sᵣₑₚ by using the plurality of 2D segmentation data Sₚ₁, Sₚ₂, . . . , Sₚₙ. In an exemplary embodiment, the segmentation map generation unit 130 generates the 3D segmentation map Sᵣₑₚ by using a back-projection algorithm for the plurality of 2D segmentation data Sₚ₁, Sₚ₂, . . . , Sₚₙ.

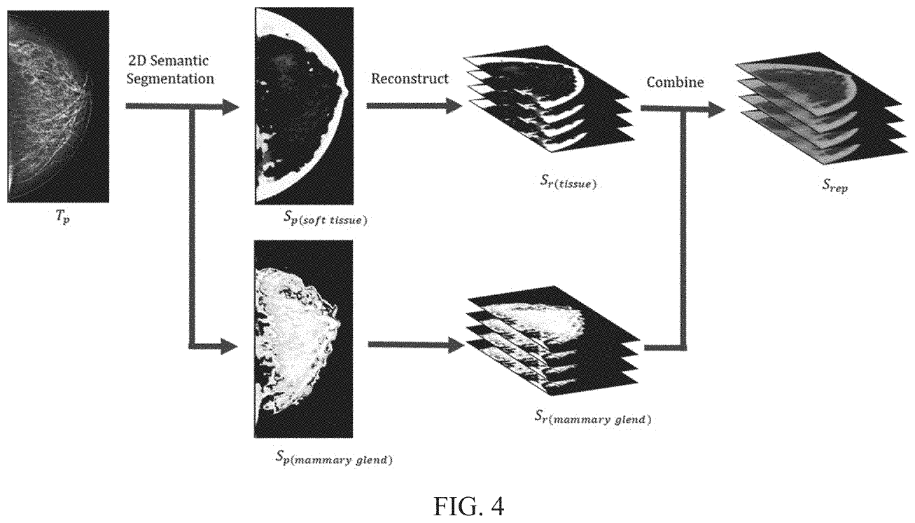

FIG. 4 is a diagram exemplarily illustrating a process of generating a segmentation map by a segmentation map generation unit in an apparatus for generating a synthetic 2D image according to an exemplary embodiment of the present invention.

The semantic segmentation is performed for the plurality of 2D images Tₚ transferred from the image input unit 110 and the 2D segmentation data Sₚ is acquired. In the case of the 2D image Tₚ, a plurality of class labels may be granted to one pixel due to characteristics of a radiographic image and the multi-label pixelwise classification is performed for the plurality of 2D images Tₚ. In the example of FIG. 4, it is illustrated that multi-label segmentation is performed with the class labels such as the tissue and the mammary gland for the 2D image Tₚ, and 2D segmentation data Sₚ₍ₜᵢₛₛᵤₑ₎ for the tissue and 2D segmentation data S_{p(mammary gland)} for the mammary gland are acquired. The plurality of 2D segmentation data Sₚ may be integrated into the 3D segmentation map Sᵣₑₚ. In an exemplary embodiment, 3D segmentation data Sᵣ₍ₜᵢₛₛᵤₑ₎ or S_{r(mammary gland)} reconstructed for each class label may be generated by using the 2D segmentation data Sₚ₍ₜᵢₛₛᵤₑ₎ or S_{p(mammary gland)} for a specific class label and the 3D segmentation map Sᵣₑₚ may be generated by using the 3D segmentation data Sᵣ₍ₜᵢₛₛᵤₑ₎ or S_{r(mammary gland)} reconstructed for each class label.

In other words, the segmentation map generation unit 130 may obtain 2D segmentation data Sₚ for each class label for each 2D image Tₚ obtained at various angles, and then generate the 3D segmentation data Sᵣ for each class label by using the plurality of 2D segmentation data Sₚ. In this case, each voxel of the 3D segmentation data Sᵣ for each class label may include a value (for example, a probability of a specific label) with respect to the degree to which the class label may be a specific class label. In an exemplary embodiment, in generating the 3D segmentation data Sᵣ, a back-projection technique may be used.

Individual pixels constituting the 2D image Tₚ may include several class labels due to superimposition, but individual voxels constituting the reconstructed 3D image Tᵣ may have one class label. For this reason, in the process of generating the 3D segmentation map Sᵣₑₚ using the 3D segmentation data Sᵣ for each class label, class labels for individual voxels are specified. In an exemplary embodiment, when a plurality of class labels exist in individual voxels of the 3D segmentation map Sᵣₑₚ, the class label of the corresponding voxel may be determined as the class label with the highest probability. For example, when there are M 3D segmentation data (Sᵣₖ, k=1 . . . M), the class label (Sᵣₑₚ(x, y, z)) of a specific voxel of the 3D segmentation map (Sᵣₑₚ) may be obtained by Equation 1.

$${S}_{rep} ( x , y , z ) = {argmax}_{k} ( {S}_{rk} ( x , y , z ) ) \\ ( k = 1 … M ) \qquad [ Equation 1 ]$$

Referring back to FIG. 2, the synthetic 2D image synthesis unit 150 generates a synthetic 2D image F_{syn} by using the 3D segmentation map Sᵣₑₚ generated by the segmentation map generation unit 130 and the reconstructed 3D image Tᵣ generated by the tomosynthesis unit 120. In this case, the synthetic 2D image synthesis unit 150 may generate synthetic 2D image F_{syn} by applying the semantic filter provided by the semantic filter unit 140.

The semantic filter unit 140 may generate the semantic filter from the 3D segmentation map Sᵣₑₚ according to the setting or request of the user. The semantic filter may be described as a weight to be described below, and a specific material may be emphasized or suppressed by assigning the weight for each material in the synthetic 2D image synthesis process. That is, the intensity of an image pixel by a specific material in the generated synthetic 2D image may be adjusted.

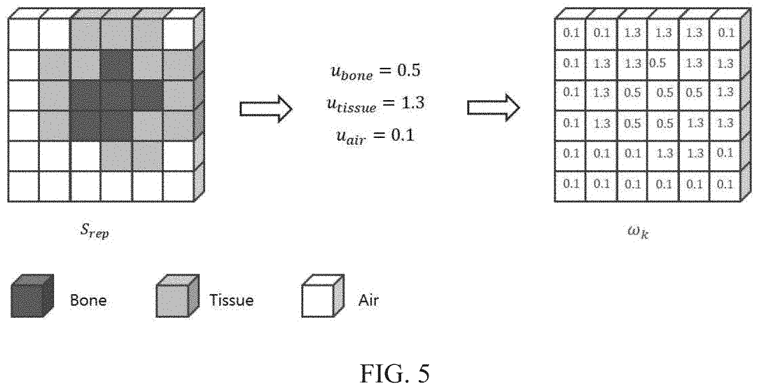

FIG. 5 is a diagram illustrating that a semantic filter is generated by a semantic filter unit in an apparatus for generating a synthetic 2D image according to an exemplary embodiment of the present invention.

In FIG. 5, a part of the 3D segmentation map Sᵣₑₚ is illustrated. The weight may be granted for each label according to the user's selection or the user's request. In FIG. 5, it is illustrated that a weight u_{bone} of the class label for the bone is set 0.5, a weight uₜᵢₛₛᵤₑ of the class label for the tissue is set to 1.3, and a weight uₐᵢᵣ of the class label for the air is set to 0.1.

By combining the weights for each label, a weight ωₖ which may be applied as the semantic filter may be acquired.

Numerical values illustrated in FIG. 5 represent a case of bone suppression in which a bone portion is weakened, and the weight of the voxel corresponding to the bone is lower than those of the others to suppress the bone portion in the synthetic 2D image.

As such, a semantic filter is generated in which the weight for the specific class label is increased or decreased, and applied to the synthetic 2D image synthesis to acquire an image in which a body tissue desired by the user is emphasized or weakened.

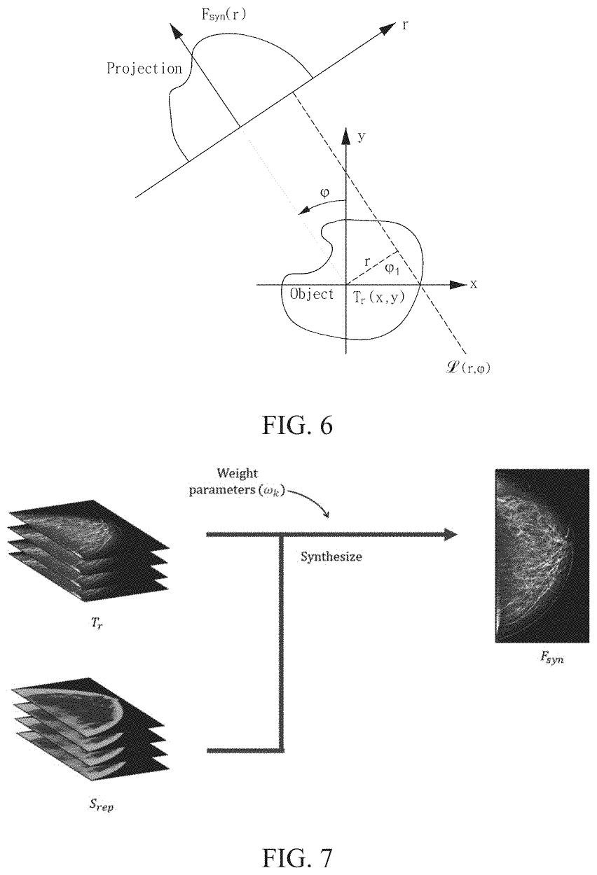

FIG. 6 is a diagram for describing that the synthetic 2D image is generated by applying the semantic filter in an apparatus for generating a synthetic 2D image according to an exemplary embodiment of the present invention and FIG. 7 is a diagram exemplarily illustrating that the synthetic 2D image is generated in an apparatus for generating a synthetic 2D image according to an exemplary embodiment of the present invention.

In FIG. 6, it is illustrated that 1D data is generated in 2D reconstruction data for convenience of understanding. That is, in the synthetic 2D image according to the present invention, the 2D data is generated in the 3D data and FIG. 6 illustrates that a dimension is lowered in order to exemplarily describe the method.

In FIG. 6, in Tᵣ in which the reconstructed object exists, projection data that may be generated through Tᵣ may be represented by F_{syn}(r). In this case, F_{syn}(r) may represent the sum of the attenuation degree when the radiation passes through the object. Therefore, when a line integral for a ray (a ray formed by a pixel of an image to be generated and the radiation source) perpendicular to a ray inclined by φ on the x axis and spaced by r from the center is represented by F_{syn}(r) in FIG. 6, the F_{syn}(r) may be shown in Equation 2.

F_{syn}(r)=∫_{-∞}^{∞}∫_{-∞}^{∞}Tᵣ(x,y)δ(x cos φ+y sin φ−r)dxdy [Equation 2]

A semantic filter for giving the weight for a specific material, i.e., a weight function ωₙ is applied to Equation 2, which is shown in Equation 3.

F_{syn}(r)=∫_{-∞}^{∞}∫_{-∞}^{∞}Tᵣ(x,y)ωₙ(x,y)δ(x cos φ+y sin φ−r)dxdy [Equation 3]

In an exemplary embodiment, when a weight set by the user for each voxel in the 3D segmentation map Sᵣₑₚ is ωₖ, ωₙ may be acquired by normalizing ωₖ according to a voxel group in a ray formed by the pixel of the image to be generated and the radiation source. Normalization may be performed by dividing a voxel to be normalized by the sum of weights of voxels in the ray formed by the radiation source.

When the weight of a specific voxel in the segmentation map Sᵣₑₚ is represented by ωₖ(x, y), the sum of ωₖ(x, y) of voxels included in a specific ray ωₖ′(r) may be expressed as in Equation 4 and the relationship between ωₙ, ωₖ, and ω_{k′} may be expressed as in Equation 5, and accordingly, Equation 3 may be expressed as Equation 6 again.

$$\begin{matrix}{ω}_{{k}^{′}} ( r ) = {\int }_{- ∞}^{∞} {\int }_{- ∞}^{∞} {ω}_{k} ( x , y ) δ ( x cos φ + y sin φ - r ) dxdy & [ Equation 4 ] \\ {ω}_{n} = \frac{{ω}_{k}}{{ω}_{{k}^{′}}} & [ Equation 5 ] \\ {F}_{syn} ( r ) = {\int }_{- ∞}^{∞} {\int }_{- ∞}^{∞} \frac{{T}_{r} ( x , y ) {ω}_{k} ( x , y ) δ ( x cos φ + y sin φ - r )}{{\int }_{- ∞}^{∞} {\int }_{- ∞}^{∞} {ω}_{k} ( x , y ) δ ( x cos φ + y sin φ - r ) dxdy} dxdy & [ Equation [ 6 ]\end{matrix}$$

Referring to FIG. 7, in the process of generating the synthetic 2D image F_{syn} using the 3D image Tᵣ reconstructed and the 3D segmentation map Sᵣₑₚ as in the above process, the weight ωₖ serves as the semantic filter. As a result, a synthetic 2D image F_{syn} in which a specific material is emphasized or suppressed—that is, the intensity of an image by a specific material is adjusted—according to the user's setting or request may be generated.

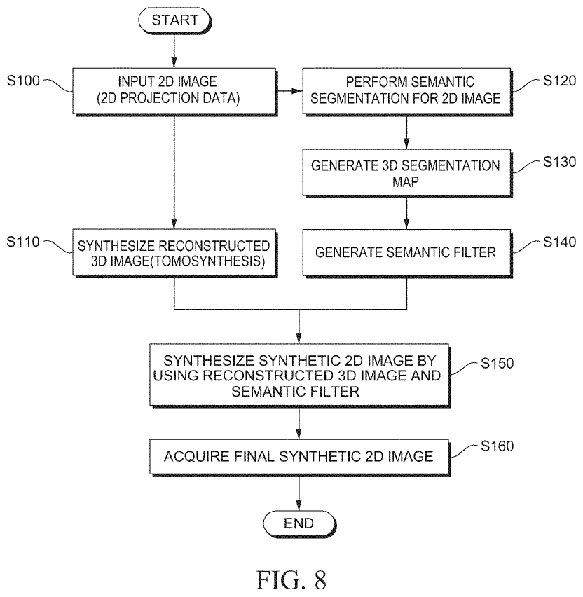

FIG. 8 is a flowchart illustrating a method for generating a synthetic 2D image according to an exemplary embodiment of the present invention.

The image input unit 110 receives the plurality of 2D images which are the 2D projection data from the detector 20 of the radiographic image acquisition apparatus 10 (S100).

The tomosynthesis unit 120 applies a tomosynthesis technique to the plurality of 2D images transferred from the image input unit 110 and reconstructs the 2D images in 3D to generate the reconstructed 3D image (S110).

The segmentation map generation unit 130 generates the 3D segmentation map for a region including the target object 18. Specifically, the segmentation map generation unit 130 performs semantic segmentation for each of the plurality of 2D images transferred from the image input unit 110 to acquire 2D segmentation data (S120).

Next, the segmentation map generation unit 130 generates a 3D segmentation map by reconstructing the 2D segmentation data in 3D (S130).

The semantic filter unit 140 generates a semantic filter as a weight to be applied to segmentation data (e.g., class label information) of each voxel of the 3D segmentation map according to the user's request or setting (S140).

The synthetic 2D image synthesis unit 150 generates a synthetic 2D image by using the reconstructed 3D image and the semantic filter (S150).

Through the above process, a final synthetic 2D image may be acquired (S160) and the final synthetic 2D image may be presented to a user through a display 160.

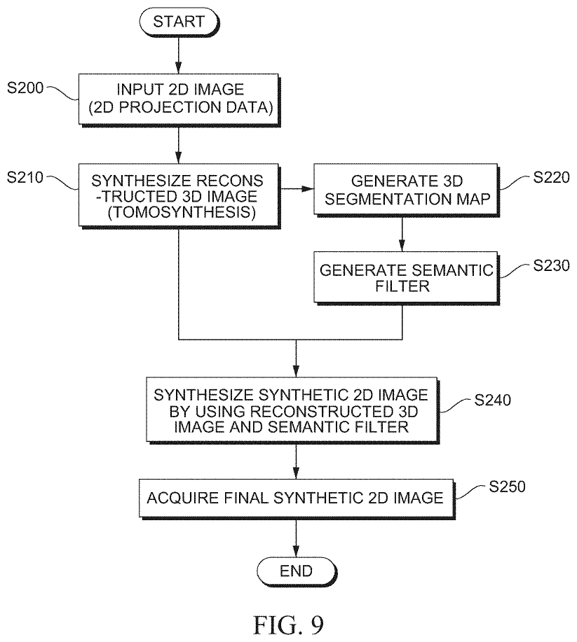

FIG. 9 is a flowchart illustrating a method for generating a synthetic 2D image according to another exemplary embodiment of the present invention.

The image input unit 110 receives the plurality of 2D images which are the 2D projection data from the detector 20 of the radiographic image acquisition apparatus 10 (S200).

The tomosynthesis unit 120 applies a tomosynthesis technique to the plurality of 2D images transferred from the image input unit 110 and reconstructs the 2D images in 3D to generate the reconstructed 3D image (S210).

The segmentation map generation unit 130 generates the 3D segmentation map by using the reconstructed 3D image (S220). In step S220, the 3D segmentation map may be generated by directly performing the semantic segmentation for each voxel of the reconstructed 3D image. As a result of performing step S220, the class label for each voxel may be assigned.

The semantic filter unit 140 generates a semantic filter as a weight to be applied to segmentation data of each voxel of the 3D segmentation map according to the user's request or setting (S230).

The synthetic 2D image synthesis unit 150 generates a synthetic 2D image by using the reconstructed 3D image and the semantic filter (S240).

Through the above process, a final synthetic 2D image may be acquired (S250) and the final synthetic 2D image may be presented to a user through a display 160.

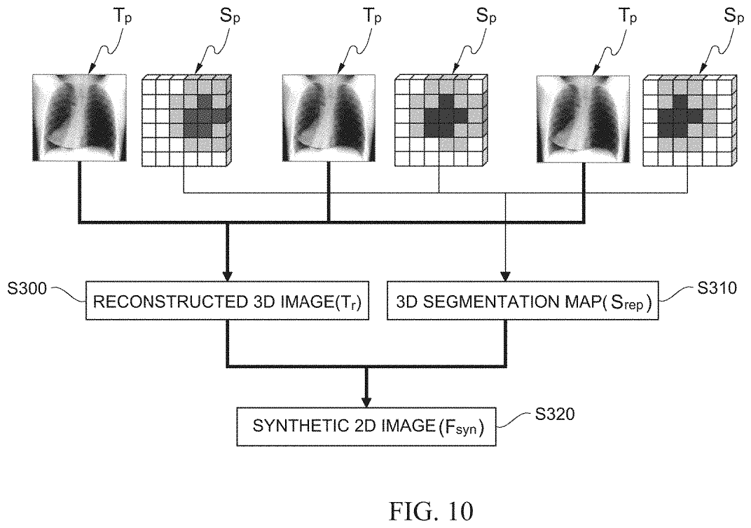

FIG. 10 is a diagram schematically illustrating a method for generating a synthetic 2D image according to an exemplary embodiment of the present invention.

A plurality of 2D images Tₚ which are 2D projection data are input from the detector 20 of the radiographic image acquisition apparatus 10 and the reconstructed 3D image Tᵣ is generated (S300).

The segmentation map generation unit 130 generates the 3D segmentation map Sᵣₑₚ (S310).

The synthetic 2D image synthesis unit 150 generates the synthetic 2D image F_{syn} by using the reconstructed 3D image Tᵣ and the 3D segmentation map Sᵣₑₚ.

According to the present invention, a filter for emphasizing information requested by the user may be dynamically generated by using semantic segmentation data. In addition, by using semantic segmentation data, even voxels having similar pixel values (pixel intensity) are classified and different weights are used for each material or characteristic are used to obtain an adaptive synthetic 2D image.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. An apparatus for generating a synthetic 2D image, the apparatus comprising:
an image input unit receiving a 2D image at a plurality of angles or locations for a target object from a detector of a radiographic image acquisition apparatus;
a tomosynthesis unit generating a reconstructed 3D image by using the 2D image input into the image input unit;
a segmentation map generation unit generating a 3D segmentation map including segmentation data indicating characteristics or types of voxels constituting the 3D image; and
a synthetic 2D image synthesis unit generating a synthetic 2D image by using the reconstructed 3D image and the 3D segmentation map.

2. The apparatus of claim 1, wherein the synthetic 2D image synthesis unit generates a synthetic 2D image in which the intensity of a specific material of the target object is adjusted by using the segmentation data.

3. The apparatus of claim 1, wherein the segmentation map generation unit assigns at least one class label as the segmentation data to the voxel.

4. The apparatus of claim 3, wherein the segmentation map generation unit receives the 2D image from the image input unit and acquires a plurality of 2D segmentation data by performing semantic segmentation in the 2D image, and generates the 3D segmentation map by using the plurality of 2D segmentation data.

5. The apparatus of claim 4, wherein the segmentation map generation unit generates the 3D segmentation map by back-projecting the plurality of 2D segmentation data.

6. The apparatus of claim 4, wherein the segmentation map generation unit performs the semantic segmentation for each class label for the 2D image to acquire 2D segmentation data for each class label.

7. The apparatus of claim 1, wherein the segmentation map generation unit generates the 3D segmentation map by performing the semantic segmentation for each voxel of the reconstructed 3D image.

8. The apparatus of claim 1, further comprising:
a semantic filter unit generating a semantic filter as a weight to be applied to the segmentation data of the voxel included in the 3D segmentation map.

9. The apparatus of claim 8, wherein the weight is set separately for each segmentation data or for each voxel.

10. The apparatus of claim 9, wherein the weight is assigned according to characteristics of the voxel or a material corresponding to the voxel, and generated through a normalization process.

11. The apparatus of claim 8, wherein the synthetic 2D image synthesis unit generates the synthetic 2D image by multiplying the intensity of the voxel by the semantic filter.

12. A method for generating a synthetic 2D image, the method comprising:
(a) receiving a 2D image at a plurality of angles or locations for a target object from a detector of a radiographic image acquisition apparatus;
(b) generating, by a tomosynthesis unit, a reconstructed 3D image by using the 2D images;
(c) generating, by a segmentation map generation unit, a 3D segmentation map including segmentation data indicating characteristics or types of voxels of the reconstructed 3D image; and
(d) generating, by a synthetic 2D image synthesis unit, a synthetic 2D image by using the reconstructed 3D image and the 3D segmentation map.

13. The method of claim 12, wherein in step (d) above, a synthetic 2D image is generated in which the intensity of at least one material of the target object is adjusted by using the segmentation data.

14. The method of claim 12, wherein in step (c), the segmentation map generation unit receives the 2D image from the image input unit and acquires a plurality of 2D segmentation data by performing semantic segmentation in the 2D image, and generates the 3D segmentation map by using the plurality of 2D segmentation data.

15. The method of claim 12, wherein in step (c), the 3D segmentation map is generated by performing the semantic segmentation for each voxel of the reconstructed 3D image.

16. The method of claim 14, wherein in step (d), a semantic filter as a weight to be applied to segmentation data of each voxel of the 3D segmentation map is generated and the synthetic 2D image is generated by using the semantic filter.

17. The method of claim 16, wherein the weight is set separately for each segmentation data or for each voxel.

18. The method of claim 17, wherein the weight is assigned according to characteristics of the voxel or a material corresponding to the voxel, and the weight is generated through a normalization process.

19. The method of claim 16, wherein in step (d), the synthetic 2D image is generated by multiplying the intensity of the voxel by the semantic filter.
